(19) United States
(12) Patent Application Publication
(10) Pub. No.: US 2007/0015750 A1
(43) Pub. Date: Jan. 18, 2007

(54) Tetrahydronaphthalene derivatives, process for their production and their use as anti-inflammatory agents

(76) Inventors: Stefan Baeurle, Berlin (DE); Heike Schaecke, Berlin (DE); Markus Berger, Berlin (DE); Anne Mengel, Berlin (DE)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 11/399,590

(22) Filed: Apr. 7, 2006

Related U.S. Application Data
(60) Provisional application No. 60/671,064, filed on Apr. 14, 2005.

(30) Foreign Application Priority Data
Apr. 14, 2005 (DE) 10 2005 017 286.5

(51) Int. Cl. A61K 31/538; A61K 31/517; A61K 31/502

(52) U.S. Cl. 514/230.5; 514/249; 514/303; 514/248; 514/266.1; 514/310; 514/312; 514/443; 514/417; 514/415; 514/300; 514/456; 514/394; 514/406; 514/639; 514/681; 544/105; 544/235; 544/237; 544/283; 544/353

(57) ABSTRACT

The invention relates to polysubstituted tetrahydronaphthalene derivatives of formula (I), 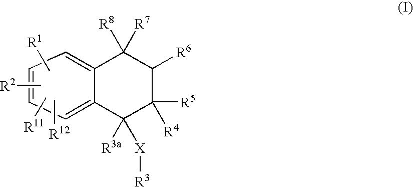 process for their production and their use as anti-inflammatory agents.

This application claims the benefit of the filing date of U.S. Provisional Application Ser. No. 60/671,064 filed Apr. 14, 2005, which is incorporated by reference herein.

### INTRODUCTION

The invention relates to tetrahydronaphthalene derivatives, process for their production and their use as anti-inflammatory agents.

Open-chain, non-steroidal anti-inflammatory agents are known from the prior art (DE 100 38 639 and WO 02/10143). In the experiment, these compounds show dissociations of action between anti-inflammatory are undesirable metabolic actions and are superior to the previously described nonsteroidal glucocorticoids or exhibit at least just as good an action.

In this invention, additional non-steroidal anti-inflammatory agents are provided.

### SHORT DESCRIPTION OF THE INVENTION

This invention relates to compounds of general formula (I), 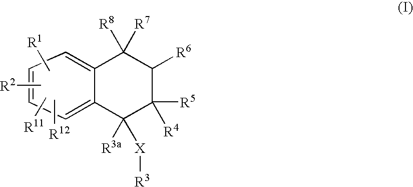
in which
R¹ and R², independently of one another, are a hydrogen atom, a hydroxy group, a halogen atom, an optionally substituted (C₁-C₁₀)-alkyl group, a (C₁-C₁₀)-alkoxy group, a (C₁-C₁₀)-alkylthio group, a (C₁-C₅)-perfluoroalkyl group, a cyano group, a nitro group, or an —NR⁹R^{9a} group,
or R¹ and R² together form a group that is selected from the groups —O—(CH₂)ₙ—O—, —O—(CH₂)ₙ—CH₂—, —O—CH═CH—, —(CH₂)ₙ₊₂—, —NH—(CH₂)ₙ₊₁—, —N(C₁-C₃-alkyl)-(CH₂)ₙ₊₁—, and —NH—N═CH—,
whereby n=1 or 2, and the terminal oxygen atoms and/or carbon atoms and/or nitrogen atoms are linked to directly adjacent ring-carbon atoms,
R¹¹ is a hydrogen atom, a hydroxy group, a halogen atom, a cyano group, an optionally substituted (C₁-C₁₀)-alkyl group, a (C₁-C₁₀)-alkoxy group, a (C₁-C₁₀)-alkylthio group, or a (C₁-C₅)-perfluoroalkyl group,
R¹² is a hydrogen atom, a hydroxy group, a halogen atom, a cyano group, an optionally substituted (C₁-C₁₀)-alkyl group, or a (C₁-C₁₀)-alkoxy group,
R³ is a (C₁-C₁₀)-alkyl group that optionally is substituted by 1 to 3 hydroxy groups, 1 to 3 halogen atoms, and/or 1 to 3 (C₁-C₅)-alkoxy groups, an optionally substituted (C₃-C₇)-cycloalkyl group,
an optionally substituted heterocyclyl group,
an optionally substituted aryl group,
a monocyclic or bicyclic heteroaryl group that optionally is substituted by one or more groups, which are selected, independently of one another, from
   (C₁-C₅)-alkyl groups, which themselves optionally can be substituted by 1 to 3 hydroxy groups or 1 to 3 —COOR¹³ groups,
   (C₁-C₅)-alkoxy groups,
   halogen atoms, hydroxy groups, —NR⁹R^{9a} groups, and
   exomethylene groups,
and that contains 1 to 4 nitrogen atoms and/or 1 to 2 oxygen atoms and/or 1 to 2 sulfur atoms and/or 1 to 2 keto groups, whereby this group is linked to the group X via any position, and optionally can be hydrogenated at one or more sites,
R^{3a} means a hydrogen atom, a hydroxy group, an —OR¹⁰ group or an —O(CO)R¹⁰ group,
R⁴ is a hydroxy group, an —OR¹⁰ group or an —O(CO)R¹⁰ group,
R⁵ is a (C₁-C₁₀)-alkyl group, which optionally is partially or completely fluorinated, a (C₃-C₇)cycloalkyl group, a (C₁-C₈)alkyl-(C₃-C₇)cycloalkyl group, a (C₂-C₈)alkenyl-(C₃-C₇)cycloalkyl group, a heterocyclyl group, a (C₁-C₈)alkylheterocyclyl group, a (C₂-C₈)-alkenylheterocyclyl group, an aryl group, a (C₁-C₈)alkylaryl group, a (C₂-C₈)alkenylaryl group, or a (C₂-C₈)alkinylaryl group;
a monocyclic or bicyclic heteroaryl group that optionally is substituted by 1 to 2 keto groups, 1 to 2 (C₁-C₅)-alkyl groups, 1 to 2 (C₁-C₅)-alkoxy groups, 1 to 3 halogen atoms, and/or 1 to 2 exomethylene groups and that contains 1 to 3 nitrogen atoms and/or 1 to 2 oxygen atoms and/or 1 to 2 sulfur atoms;
a (C₁-C₈)alkylheteroaryl group, a (C₂-C₈)alkenylheteroaryl group, or a (C₂-C₈)alkinylheteroaryl group,
whereby this group is linked to the tetrahydronaphthalene system via any position, and optionally can be hydrogenated at one or more sites,
R⁶ is a hydrogen atom, a halogen atom, or an optionally substituted (C₁-C₁₀)-alkyl group,
R⁷ and R⁸, independently of one another, mean a hydrogen atom, a halogen atom, an optionally substituted (C₁-C₁₀)-alkyl group, a cyano group, together a (C₁-C₁₀)-alkylidene group or together with the carbon atom of the tetrahydronaphthalene system an optionally substituted (C₃-C₆)-cycloalkyl ring; or
R⁶ and R⁷ together form an annelated five- to eight-membered, saturated or unsaturated carbocyclic compound or heterocyclic compound, which optionally is substituted by 1 to 2 keto groups, 1 to 2 (C₁-C₅)-alkyl groups, 1 to 2 (C₁-C₅)-alkoxy groups, and/or 1 to 4 halogen atom; or
R¹ and R⁸ together form an annelated five- to eight-membered, saturated or unsaturated carbocyclic compound or heterocyclic compound, which optionally is substituted by 1 to 2 keto groups, 1 to 2 (C₁-C₅)-alkyl groups, 1 to 2 (C₁-C₅)-alkoxy groups, and/or 1 to 4 halogen atoms;
R⁹ and R^{9a}, independently of one another, mean a hydrogen atom, (C₁-C₅)-alkyl or —(CO)—(C₁-C₅)-alkyl,
R¹⁰ means a (C₁-C₁₀)-alkyl group or any hydroxy protective group,
R¹³ means a hydrogen atom or a (C₁-C₅)-alkyl group, and
X means a bond or a group —C(═O)—, —C(═S)—, —O—C(═O)—, —O—C(═O)—O—, —O—C(═O)—NH—, —(CH₂)ₚ— (whereby p=1, 2 or 3), a group —(CH₂)ₚ—NH— (whereby p=1, 2 or 3) or a group —NH—, whereby if X contains an NH group, this NH group is connected to substituent R³;

with the condition that if X represents a group —NH—, R^{3a} is not a hydrogen atom; in the form of any stereoisomer or a mixture of stereoisomers, or as a pharmacologically harmless salt or derivative.

In addition, this invention relates to processes for the production of compounds of general formula (I), as described herein.

In addition, this invention relates to pharmaceutical compositions that comprise one or more compounds of general formula (I) in combination with one or more pharmaceutical vehicles and/or adjuvants.

This invention relates, moreover, to the use of compounds of general formula (I) for the production of pharmaceutical compositions with anti-inflammatory action

This invention relates, moreover, to compounds of general formula (II), 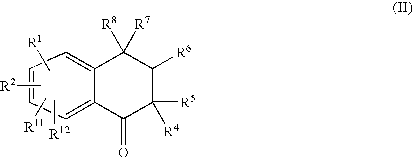
in which substituents R¹ to R¹² have the above-indicated meanings, as well as the use of these compounds for the production of compounds of general formula (I).

### IN-DEPTH DESCRIPTION OF THE INVENTION

Definitions

The designation halogen atom or halogen means a fluorine, chlorine, bromine or iodine atom. A fluorine, chlorine or bromine atom is preferred.

The alkyl groups that are mentioned in the definitions of general formula (I) can be straight-chain or branched and stand for, for example, a methyl, ethyl, n-propyl, iso-propyl, n-butyl, iso-butyl, tert-butyl or n-pentyl, 2,2-dimethylpropyl, 2-methylbutyl or 3-methylbutyl group, as well as the hexyl, heptyl, nonyl, or decyl group and any branched derivatives thereof. Alkyl groups that contain 1 to 10, 1 to 8, or 1 to 5 carbon atoms are preferred. A methyl or ethyl group is especially preferred.

The above-mentioned alkyl groups optionally can be substituted by 1 to 5, preferably 1 to 3 groups, which are selected, independently of one another, from hydroxy, cyano, nitro, —COOR¹³, C₁-C₅-alkoxy groups, halogen, —NR⁹R^{9a}, and a partially or completely fluorinated C₁-C₃-alkyl group. The alkyl groups preferably can be substituted by 1 to 3 halogen atoms and/or 1 to 3 hydroxy and/or 1 to 3 cyano and/or 1 to 3 —COOR¹³ groups. Fluorine atoms, and hydroxy, methoxy and/or cyano groups represent an especially preferred subgroup of substituents.

1 to 3 hydroxy groups and/or 1 to 3 —COOR¹³ groups are another preferred group of substituents for the alkyl groups. In this case, the hydroxy groups are especially preferred.

For a partially or completely fluorinated alkyl group, for example, the following partially or completely fluorinated groups are considered: fluoromethyl, difluoromethyl, trifluoromethyl, fluoroethyl, 1,1-difluoroethyl, 1,2-difluoroethyl, 1,1,1-trifluoroethyl, tetrafluoroethyl, and pentafluoroethyl. Of the latter, the trifluoromethyl or pentafluoroethyl group is preferred. The completely fluorinated group is also named a perfluoroalkyl group. The reagents that optionally are used during synthesis are commercially available or the published syntheses of the corresponding reagents are part of the prior art, or published syntheses can be used analogously.

The alkenyl groups have at least one C═C-double bond and can be straight-chain or branched. Alkenyl groups with 2 to 8 carbon atoms are preferred.

The alkinyl groups have at least one C≡C-triple bond and can be straight-chain or branched. Alkinyl groups with 2 to 8 carbon atoms are preferred.

The alkoxy groups that are mentioned in the definitions of general formula (I) can be straight-chain or branched and stand for, for example, a methoxy, ethoxy, n-propoxy, iso-propoxy-, n-butoxy, iso-butoxy, tert.-butoxy or n-pentoxy, 2,2-dimethylpropoxy, 2-methylbutoxy or 3-methylbutoxy group. C₁-C₅— as well as C₁-C₃—, C₁-C₈—, and C₁-C₁₀-alkoxy groups are preferred. A methoxy or ethoxy group is especially preferred.

The alkylthio groups that are mentioned in the definitions of general formula (I) can be straight-chain or branched and stand for, for example, a methylthio, ethylthio, n-propylthio, iso-propylthio, n-butylthio, iso-butylthio, tert.-butylthio or n-pentylthio, 2,2-dimethylpropylthio, 2-methylbutylthio or 3-methylbutylthio group. C₁-C₅-Alkylthio groups are preferred A methylthio or ethylthio group is especially preferred

The above-described alkoxy and alkylthio groups can carry the same substituents on their alkyl groups that were already described above for alkyl groups in general. Preferred substituents for alkoxy and alkylthio groups are selected independently of one another from halogen (in particular fluorine and/or chlorine), hydroxy and cyano.

Substituent —NR⁹R^{9a} means, for example, —NH₂, —NH(CH₃), —N(CH₃)₂, —NH(C₂H₅), —N(C₂H₅)₂, —NH(C₃H₇), —N(C₃H₇)₂, —NH(C₄H₉), —N(C₄H₉)₂, —NH(C₅H₁₁), —N(C₅H₁₁)₂, —NH(CO)CH₃, —NH(CO)C₂H₅, —NH(CO)C₃H₇, —NH(CO)C₄H₉, or —NH(CO)C₅H₁₁.

The (C₃-C₇)-cycloalkyl group means a saturated cyclic group with 3 to 7 ring-carbon atoms that optionally is substituted by one or more groups, selected from hydroxy groups, halogen atoms, (C₁-C₅)-alkyl groups, (C₁-C₅)-alkoxy groups, —NR⁹R^{9a} groups, —COOR¹³ groups, —CHO and cyano, such as, for example, cyclopropyl, methylcyclopropyl, cyclobutyl, methylcyclobutyl, cyclopentyl, methylcyclopentyl, cyclohexyl, methylcyclohexyl, cycloheptyl, and methylcycloheptyl.

A (C₁-C₈)alkyl-(C₃-C₇)cycloalkyl group R⁵ is defined as a cycloalkyl group (as defined above) that is linked to the ring system via a straight-chain or branched (C₁-C₈)-alkyl unit (as defined above). Examples of such groups are —(CH₂)-cycloalkyl, —(C₂H₄)-cycloalkyl, —(C₃H₆)-cycloalkyl, —(C₄H₈)-cycloalkyl, and —(C₅H₁₀)-cycloalkyl, whereby cycloalkyl is defined as described above.

A (C₂-C₈)alkenyl-(C₃-C₇)cycloalkyl group R⁵ is defined as a cycloalkyl group (as defined above) that is linked to the ring system via a straight-chain or branched (C₂-C₈)-alkenyl unit. Examples of such groups are —(CH═CH)-cycloalkyl, —[C(CH₃)═CH]-cycloalkyl, —[CH═C(CH₃)]-cycloalkyl, —(CH═CH—CH₂)-cycloalkyl, —(CH₂—CH═CH)-cycloalkyl, —(CH═CH—CH₂—CH₂)-cycloalkyl, —(CH₂—CH═CH—CH₂)-cycloalkyl, —(CH₂—CH₂—CH═CH)-cycloalkyl, —(C(CH₃)═CH—CH₂)-cycloalkyl, and —(CH═C(CH₃)—CH₂)-cycloalkyl.

A group with 1 to 10 carbon atoms that is bonded to the system (ring or chain) via an exo-double bond is defined as an alkylidene or exoalkylidene group. (C₁-C₅)— and (C₁-C₃)-alkylidene are preferred; exomethylene is especially preferred.

The heterocyclyl group is a cyclic, non-aromatic group that contains one or more heteroatoms and can be, for example, pyrrolidine, imidazolidine, pyrazolidine or piperidine. Perhydroquinoline and perhydroisoquinoline also belong to the heterocyclyl groups according to the invention.

As substituents for heterocyclyl and heteroaryl groups, for example, substituents from the following groups are considered: optionally substituted (C₁-C₅)-alkyl group, hydroxy, (C₁-C₅)-alkoxy, —NR⁹R^{9a}, halogen, cyano, —COOR¹³, and —CHO. The substituents optionally also can be bonded to the nitrogen atom of the heterocyclyl or heteroaryl group, N-oxides are also included in the definition.

For the purposes of the invention, aryl groups are aromatic or partially aromatic carbocyclic groups with 6 to 14 carbon atoms that have a ring, such as, e.g., phenyl or phenylene or several condensed rings, such as, e.g., naphthyl or anthranyl. By way of example, phenyl, naphthyl, tetralinyl, anthranyl, indanyl, and indenyl can be mentioned. The optionally substituted phenyl group and the naphthyl group are preferred.

At any suitable site that results in a stable compound, the aryl groups can be substituted by one or more radicals from the group of hydroxy, halogen, or (C₁-C₅)-alkyl, (C₁-C₅)-alkoxy, cyano, —CF₃, and nitro that optionally is substituted by 1 to 3 hydroxy groups or —COOR¹³ groups.

The aryl groups can be partially hydrogenated and then can also carry keto groups or exoalkylidene groups in addition to or as an alternative to the above-cited substituents.

Partially hydrogenated phenyl is defined as, e.g., cyclohexadienyl, cyclohexenyl, or cyclohexyl. A partially hydrogenated, substituted naphthalene system is, for example, 1-tetralone or 2-tetralone.

A (C₁-C₈)alkylaryl group is an aryl group, as it is already described above, which is linked to the ring system via a straight-chain or branched (C₁-C₈)-alkyl unit (as defined above).

A (C₂-C₈)alkenylaryl group is an aryl group, as it is already described above, which is linked to the ring system via a straight-chain or branched (C₂-C₈)-alkenyl unit (as defined above).

A (C₂-C₈)alkinylaryl group is an aryl group, as it is already described above, which is linked to the ring system via a straight-chain or branched (C₂-C₈)-alkinyl unit (as defined above).

The monocyclic or bicyclic heteroaryl group optionally can contain 1 to 9 groups, selected from nitrogen atoms, oxygen atoms, sulfur atoms or keto groups, including a maximum of 4 nitrogen atoms, a maximum of 2 oxygen atoms, a maximum of 2 sulfur atoms and/or a maximum of 2 keto groups. Any subcombination of these groups is possible. The heteroaryl group can be hydrogenated at one or more sites.

Monocyclic heteroaryl groups can be, for example, pyridine, pyrazine, pyrimidine, pyridazine, triazine, azaindolizine, 2H— and 4H-pyran, 2H— and 4H-thiopyran, furan, thiophene, 1H— and 4H-pyrazole, 1H— and 2H-pyrrole, oxazole, thiazole, furazan, 1H— and 4H-imidazole, isoxazole, isothiazole, oxadiazole, triazole, tetrazole, or thiadiazole.

Bicyclic heteroaryl groups can be, for example, phthalidyl, thiophthalidyl, indolyl, isoindolyl, dihydroindolyl, dihydroisoindolyl, indazolyl, benzothiazolyl, indolonyl, dihydroindolonyl, isoindolonyl, dihydroisoindolonyl, benzofuranyl, benzo[b]thienyl, benzo[c]thienyl, benzimidazolyl, dihydroisoquinolinyl, dihydroquinolinyl, benzoxazinonyl, phthalazinonyl, dihydrophthalazinonyl, quinolinyl, isoquinolinyl, quinolonyl, isoquinolonyl, quinazolinyl, quinoxalinyl, cinnolinyl, phthalazinyl, dihydrophthalazinyl, 1,7- or 1,8-naphthyridinyl, counarinyl, isocoumarinyl, indolizinyl, isobenzofuranyl, azaindolyl, azaisoindolyl, furanopyridyl, furanopyrimidinyl, furanopyrazinyl, furanopyidazinyl, pyrazolo[1,5-a]pyridinyl, dihydrobenzofuranyl, dihydrofuranopyridyl, dihydrofuranopyrimidinyl, dihydrofuranopyrazinyl, dihydrofuranopyridazinyl, or dihydrobenzofuranyl group.

If the heteroaryl groups are partially or completely hydrogenated, compounds of general formula (I), in which R³ means tetrahydropyranyl, 2H-pyranyl, 4H-pyranyl, piperidyl, tetrahydropyridyl, dihydropyridyl, 1H-pyridin-2-onyl, 1H-pyridin-4-onyl, 4-aminopyridyl, 1H-pyridin-4-ylidenaminyl, chromanyl, isochromanyl, thiochromanyl, decahydroquinolinyl, tetrahydroquinolinyl, dihydroquinolinyl, 5,6,7,8-tetrahydro-1H-quinolin-4-onyl, decahydroisoquinolinyl, tetrahydroisoquinolinyl, dihydroisoquinolinyl, 3,4-dihydro-2H-benz[1,4]oxazinyl, 1,2-dihydro[1,3]benzoxazin-4-onyl, 3,4-dihydrobenz[1,4]oxazin-4-onyl, 3,4-dihydro-2H-benzo[1,4]thiazinyl, 4H-benzo[1,4]thiazinyl, 1,2,3,4-tetrahydroquinoxalinyl, 1H-cinnolin-4-onyl, 3H-quinazolin-4-onyl, 1H-quinazolin-4-onyl, 3,4-dihydro-1H-quinoxalin-2-onyl, 2,3-1,2,3,4-tetrahydro[1,5]naphthyridinyl, dihydro-1H-[1,5]naphthyridinyl, 1H-[1,5]naphthyrid-4-onyl, 5,6,7,8-tetrahydro-1H-naphthyridin-4-onyl, 1,2-dihydropyrido[3,2-d][1,3]oxazin-4-onyl, octahydro-1H-indolyl, 2,3-dihydro-1H-indolyl, octahydro-2H-isoindolyl, 1,3-dihydro-2H-isoindolyl, 1,2-dihydroindazolyl, 1H-pyrrolo[2,3-b]pyridyl, 2,3-dihydro-1H-pyrrolo[2,3-b]pyridyl, or 2,2-dihydro-1H-pyrrolo[2,3-b]pyridin-3-onyl, are part of this invention.

The monocyclic or bicyclic heteroaryl group optionally can be substituted by one or more substituents, selected from (C₁-C₅)-alkyl groups, (C₁-C₅)-alkoxy groups, halogen atoms, and/or exomethylene groups that optionally are substituted by 1 to 3 hydroxy groups and/or 1 to 3 —COOR¹³ groups. The substituents can, if possible, optionally also be directly bonded to the heteroatom (e.g., to the nitrogen atom). N-Oxides are also part of this invention.

A (C₁-C₈)alkylheteroaryl group is a heteroaryl group, as it is already described above, which is linked to the ring system via a straight-chain or branched (C₁-C₈)-alkyl unit (as defined above).

A (C₂-C₈)alkenylheteroaryl group is a heteroaryl group, as it is already described above, which is linked to the ring system via a straight-chain or branched (C₂-C₈)-alkenyl unit (as defined above).

A (C₂-C₈)alkinylheteroaryl group is a heteroaryl group, as it is already described above, which is linked to the ring system via a straight-chain or branched (C₂-C₈)-alkinyl unit (as defined above).

A (C₁-C₈)alkylheterocyclyl group is a heterocyclyl group, as it is already described above, which is linked to the ring system via a straight-chain or branched (C₁-C₈)-alkyl unit (as defined above).

A (C₂-C₈)alkenylheterocyclyl group is a heterocyclyl group, as it is already described above, which is linked to the ring system via a straight-chain or branched (C₂-C₈)-alkenyl unit (as defined above).

As hydroxy protective groups, all hydroxy protective groups that are commonly known to one skilled in the art, especially silyl ether or ester of organic C₁-C₁₀ acids, C₁-C₅-ether, benzyl ether or benzyl ester, are suitable. The commonly used hydroxy protective groups are described in detail in T. W. Greene, P. G. M. Wuts “Protective Groups in Organic Synthesis, ” 2nd Edition, John Wiley & Sons, 1991. The protective groups are preferably alkyl, aryl or mixed alkylaryl-substituted silyl groups, e.g., the trimethylsilyl (TMS), triethylsilyl (TES), tert.-butyldimethylsilyl (TBDMS), tert.-butyldiphenylsilyl (TBDPS) or triisopropylsilyl groups (TIPS) or other common hydroxy protective groups (e.g., methoxymethyl, methoxyethoxymethyl, ethoxyethyl, tetrahydrofuranyl or tetrahydropyranyl groups).

The compounds of general formula (I) according to the invention can be present as stereoisomers because of the presence of asymmetry centers. All possible diastereomers both as racemates and in enantiomer-pure form are subjects of this invention. The term stereoisomers also comprises all possible diastereomers and regioisomers and tautomers (e.g., keto-enol tautomers), in which the stereoisomers according to the invention can be present, which thus are also a subject of the invention.

The compounds according to the invention can also be present in the form of salts with pharmacologically harmless anions, for example in the form of hydrochloride, sulfate, nitrate, phosphate, pivalate, maleate, fumarate, tartrate, benzoate, mesylate, citrate or succinate.

Pharmacologically harmless derivatives or prodrugs of the compounds of general formula (I) are also comprised by the invention. As derivatives or prodrugs, for example, ester, ethers or amides of the compounds of general formula (I) or other compounds that metabolize in the organism to compounds of general formula (I) are designated. Suitable compounds are cited, for example, in Hans Bundgaard (publisher), Design of Prodrugs, Elsevier, Amsterdam 1985.

Preferred Embodiments

A subgroup of the compounds of general formula (I) according to the invention are those compounds in which R⁷ and R⁸, independently of one another, mean a hydrogen atom, a halogen atom, an optionally substituted (C₁-C₁₀)-alkyl group, a cyano group, together a (C₁-C₁₀)-alkylidene group or, together with the carbon atom of the tetrahydronaphthalene system, an optionally substituted (C₃-C₆)-cycloalkyl ring.

Another subgroup of compounds of general formula (I) according to the invention are those compounds in which R⁶ and R⁷ together form an annelated five- to eight-membered, saturated or unsaturated carbocyclic or heterocyclic compound, which optionally is substituted by 1 to 2 keto groups, 1 to 2 (C₁-C₅)-alkyl groups, 1 to 2 (C₁-C₅)-alkoxy groups, and/or 1 to 4 halogen atoms.

Another subgroup of the compounds of general formula (I) according to the invention are those compounds in which R¹ and R⁸ together form an annelated five- to eight-membered, saturated or unsaturated carbocyclic compound or heterocyclic compound, which optionally is substituted by 1 to 2 keto groups, 1 to 2 (C₁-C₅)-alkyl groups, 1 to 2 (C₁-C₅)-alkoxy groups, and/or 1 to 4 halogen atoms.

Another subgroup of the compounds of general formula (I) according to the invention are those compounds in which R¹ and R², independently of one another, are a hydrogen atom, a hydroxy group, a halogen atom, an optionally substituted (C₁-C₁₀)-alkyl group, a (C₁-C₁₀)-alkoxy group, a (C₁-C₁₀)-alkylthio group, a (C₁-C₅)-perfluoroalkyl group, a cyano group, a nitro group or an —NR⁹R^{9a} group.

Another subgroup of the compounds of general formula (I) according to the invention are those compounds in which R¹ and R² together form a group that is selected from the groups —O—(CH₂)ₙ—O—, —O—(CH₂)ₙ—CH₂—, —O—CH═CH—, —(CH₂)ₙ₊₂—, —NH—(CH₂)ₙ₊₁—, —N(C₁-C₃-alkyl)-(CH₂)ₙ₊₁—, and —NH—N═CH—, whereby n=1 or 2, and the terminal oxygen atoms and/or carbon atoms and/or nitrogen atoms are linked to directly adjacent ring-carbon atoms.

A preferred group of compounds of general formula (I) are those compounds in which X is a bond or a group —C(═O)—, —C(═S)—, —(CH₂)ₚ— (whereby p=1 or 2), or a group —(CH₂)ₚ—NH— (whereby p=1 or 2).

Another preferred group of the compounds of general formula (I) are those compounds in which R⁴ is a hydroxy group or a group —OR¹⁰.

Another preferred group of compounds of general formula (I) are those compounds in which R^{3a} is a hydroxy group, a group —OR¹⁰ or a group —O(C═O)—R¹⁰. In this connection, those compounds are especially preferred in which R¹⁰ is a (C₁-C₅)-alkyl group.

Another preferred group of compounds of general formula (I) are those compounds in which R⁵ represents a (C₁-C₅)-alkyl group or a partially or completely fluorinated (C₁-C₅)-alkyl group, an aryl group, a (C₁-C₈)alkylaryl group, a (C₂-C₈)alkenylaryl group, a (C₃-C₇)cycloalkyl group, a (C₁-C₈)alkyl(C₃-C₇)cycloalkyl group or a (C₂-C₈)alkenyl(C₃-C₇)cycloalkyl group. More preferred in this case are those compounds in which R⁵ represents a (C₁-C₅)-alkyl group or a partially or completely fluorinated (C₁-C₅)-alkyl group. Especially preferred in this case are those compounds in which R⁵ represents a trifluoromethyl group or a pentafluoroethyl group.

Another preferred group of compounds of general formula (I) are those compounds in which R⁶ is a hydrogen atom.

Another preferred group of compounds of general formula (I) are those compounds in which R⁶ is a halogen atom or an optionally substituted (C₁-C₁₀)-alkyl group.

Another preferred group of compounds of general formula (I) are those compounds in which R⁷ represents a halogen atom or an optionally substituted methyl or ethyl group.

Another preferred group of compounds of general formula (I) are those compounds in which R⁷ and R⁸ in each case represent a methyl group, or together with the carbon atom of the tetrahydronaphthalene system form a cyclopropyl group.

Another preferred group of compounds of general formula (I) are those compounds in which R³ means an optionally substituted aryl or heteroaryl group. Especially preferred in this case are those compounds in which the aryl group or heteroaryl group is selected from the group that consists of naphthyl, benzofuranyl, pyrazolo[1,5-a]pyridinyl, phenyl, phthalidyl, isoindolyl, dihydroindolyl, dihydroisoindolyl, dihydroisoquinolinyl, thiophthalidyl, benzoxazinonyl, phthalazinonyl, quinolinyl, isoquinolinyl, chromanyl, isochromanyl, quinolonyl, isoquinolonyl, indazolyl, benzothiazolyl, quinazolinyl, quinoxalinyl, cinnolinyl, phthalazinyl, 1,7- or 1,8-naphthyridinyl, dihydroindolonyl, dihydroisoindolonyl, benzimidazole or indolyl. Especially preferred in this case are the groups naphthyl, benzofuranyl, quinoxalinyl, and pyrazolo[1,5-a]pyridinyl.

Another preferred group of compounds of general formula (I) are those compounds in which substituents R¹¹ and R¹² in each case represent a hydrogen atom.

Any other possible combination of the above-mentioned subgroups and the substituents with their general and/or special meanings that are indicated as preferred can also be considered to be included by this invention.

Production Process

The compounds of general formula (I) according to the invention are available in various ways. The production processes that are described below also form a portion of this invention.

If nothing else is indicated, the substituents that are used in the process descriptions below have the same meaning as above in the section “Short Description of the Invention,” including the definitions indicated in the section “In-Depth Description of the Invention.”

In general, it is possible to use compounds of general formulas (II) and (III) for synthesis of compounds of general formula (I). These precursor compounds are produced by open-chain carbonyl compounds of general formula (IV) optionally being cyclized to compounds of general formula (III) optionally with the addition of inorganic or organic acid or Lewis acid. The latter can be oxidized to compounds of general formula (II) by the oxidation process that is known in the prior art.

The formulas above and the above-described synthesis stages are combined in Diagram 1.

The carbonyl compounds of general formula (IV) can be shown according to the instructions indicated in WO 03/082827 and WO 2005/003098.
[DIAGRAM 1: Visualization of Compounds of General Formulas (II) and (III)]

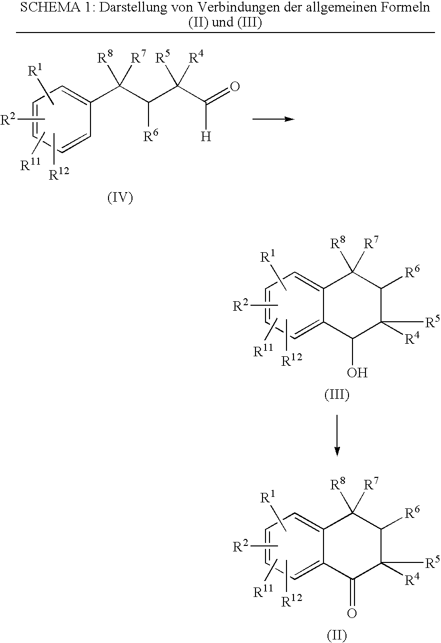

The oxidation of compounds of general formula (III) to the ketones of general formula (II) can be achieved by, for example, sulfur trioxide/pyridine complex as an oxidizing agent. In principle, however, other oxidation processes are also suitable with which secondary alcohols can be converted into ketones.

For synthesis of compounds of general formula (I), in which R^{3a} is a hydrogen atom and X represents a group —O—C(═O)—, —O—C(═O)—O— or —O—C(═O)—NH— (process A), compounds of general formula (III) are reacted with compounds of general formula R³—X-Nu (whereby Nu represents a nucleofuge group) or R³—N═C═O. As nucleofuge groups Nu in the compound R³—X-Nu, for example, halogen atoms or leaving groups, such as the acetate, tosylate, mesylate or triflate group, are suitable. The compounds R³—X-Nu thus belong to, for example, the classes of the carboxylic acid halides or the mixed anhydrides of these acids, as well as to the esters of chloroformic acid.

For synthesis of the compounds of general formula (I), in which R^{3a} means a hydroxy group, an —OR¹⁰ group or an —O(CO)R¹⁰ group (process B), a compound of general formula (II)
a) is reacted with nucleophilic reagents of general formula R³—X-M, and hydroxy group R^{3a} that is produced is optionally further modified; or
b) is reacted with nucleophilic reagents of general formula (reactive group)-M, and the (reactive group) is converted by further reactions into a group R³—X—, and hydroxy group R^{3a} that is produced is optionally further modified; or
c) is reacted with nucleophilic reagents of general formula R³—NH₂, and hydroxy group R^{3a} that is produced is optionally further modified;
   whereby substituents R¹ to R¹² and X have the above-indicated meanings, and whereby M is an electrophilic leaving group.

In the above-described process alternatives a) and b), M means an electrophilic leaving group, such as, for example, hydrogen, or a suitable main or transition group metal. Suitable metals are, for example, lithium, magnesium, silicon, copper, zinc or titanium. In addition to the above-indicated substituents R³—X— or (reactive group)-, the metals can carry still other substituents. These compounds are known to one skilled in the art.

As nucleophilic reagents R³—X-M in alternate process a) as described above, for example, organometallic compounds such as lithium, magnesium or copper-organic compounds are suitable. In these reagents, X preferably means a bond, a group —(CH₂)ₚ—, or a group —C(═O)— (which optionally is converted into a dithiane or dithiolane group for the execution of this reaction and is later deprived of protection).

As nucleophilic reagents (reactive group)-M in alternative process b) as described above, in particular organometallic vinyl, allyl or alkinyl compounds are suitable. The conversion of these reactive groups into group R³—X— can take place in a variety of ways. Thus, the unsaturated vinyl, allyl or alkinyl radicals can be used in, for example, cycloaddition reactions, with which a radical R³ can be built up. X then preferably has the meaning of a bond or a group —(CH₂)ₚ—.

As an alternative, the unsaturated vinyl, allyl or alkinyl radicals can be converted by oxidation (e.g., by ozonolysis) into the corresponding aldehydes or carboxylic acids. The latter can also be used as reactive groups for the building of a radical R³. As an alternative, an aldehyde that is obtained in this way can be reacted with a compound of general formula R³—NH₂ to form imine and then reduced. In this way, compounds are accessible in which the linker represents a group —(CH₂)ₚ—NH—.

Finally, the building of group R³—X— in the compounds of general formula (I), as described above, can also be followed by the step of benzylic dehydroxylation. In this case, hydroxy group R^{3a} is replaced by a hydrogen atom according to the process that is known in the prior art. This step is suitable for both process alternatives a) and b) of process B, as described above. For benzylic dehydrogenation, for example, the reaction with triethylsilane/boron trifluoride-etherate (Chem. Eur. J. 2001, 7, 993-1005) is suitable.

In the reaction of compounds of general formula (II) with primary amines R³—NH₂, compounds of general formula (I) are directly obtained, whereby X represents an —NH group.

Biological Activity

The anti-inflammatory action of the compounds of general formula (I) is tested in the animal experiment by tests in the croton oil-induced inflammation in rats and mice (J. Exp. Med. (1995), 182, 99-108). To this end, croton oil in ethanolic solution is applied topically to the animals' ears. The test substances are also applied topically or systemically at the same time or two hours before the croton oil. After 16-24 hours, the ear weight is measured as a yardstick for inflammatory edema, the peroxidase activity as a yardstick for the invasions of granulocytes, and the elastase activity as a yardstick for the invasion of neutrophilic granulocytes. In this test, the compounds of general formula (I) inhibit the three above-mentioned inflammation parameters both after topical administration and after systemic administration.

The binding of the substances to the glucocorticoid receptor (GR) and other steroid-hormone receptors (mineral corticoid receptor (MR), progesterone receptor (PR) and androgen receptor (AR)) is examined with the aid of recombinantly produced receptors. Cytosol preparations of Sf9 cells, which had been infected with recombinant baculoviruses that code for the GR, are used for the binding studies. In comparison to the reference substance [³H]-dexamethasone, the substances show a high affinity to the GR.

The GR-mediated inhibition of the transcription of cytokines, adhesion molecules, enzymes and other pro-inflammatory factors is considered to be an essential, molecular mechanism for the anti-inflammatory action of glucocorticoids. This inhibition is produced by an interaction of the GR with other transcription factors, eg., AP-1 and NF-kappa-B (for a survey, see Cato, A. C. B. and Wade, E., BioEssays 18, 371-378, 1996).

The compounds of general formula (I) according to the invention inhibit the secretion of cytokine IL-8 into the human monocyte cell line THP-1 that is triggered by lipopolysaccharide (LPS). The concentration of the cytokines was determined in the supernatant by means of commercially available ELISA kits.

One of the most frequent undesirable actions of a glucocorticoid therapy is the so-called “steroid diabetes” [cf., Hatz, H. J., Glucocorticoide: Immunologische Grundlagen, Pharmakologie und Therapierichtlinien, [Glucocorticoids: Immunological Bases, Pharmacology and Therapy Guidelines], Wissenschaftliche Verlagsgesellschaft mbH, Stuttgart, 1998]. The reason for this is the stimulation of gluconeogenesis in the liver by induction of the enzymes responsible in this respect and by free amino acids, which are produced from the degradation of proteins (catabolic action of glucocorticoids). A key enzyme of the catabolic metabolism in the liver is tyrosinamino transferase (TAT). The activity of this enzyme can be determined from liver homogenates by photometry and represents a good measurement of the undesirable metabolic actions of glucocorticoids. To measure the TAT induction, the animals are sacrificed 8 hours after the test substances are administered, the livers are removed, and the TAT activity is measured in the homogenate. In this test, at doses in which they have an anti-inflammatory action, the compounds of general formula (I) induce little or no tyrosinamino transferase.

Medical Indications

Because of their anti-inflammatory action, and, in addition, anti-allergic, immunosuppressive and antiproliferative action, the compounds of general formula (I) according to the invention can be used as medications for treatment or prophylaxis of the following pathologic conditions in patients, in particular mammals and preferably humans.

In this case, the term “DISEASE” stands for the following indications:
(i) Lung diseases that are accompanied by inflammatory, allergic and/or proliferative processes:
   Chronic, obstructive lung diseases of any origin, primarily bronchial asthma
   Bronchitis of different origins
   All forms of restrictive lung diseases, primarily allergic alveolitis
   All forms of pulmonary edema, primarily toxic pulmonary edema
   Sarcoidoses and granulomatoses, especially Boeck's disease
(ii) Rheumatic diseases/autoimmune diseases/joint diseases that are accompanied by inflammatory, allergic and/or proliferative processes:
   All forms of rheumatic diseases, especially rheumatoid arthritis, acute rheumatic fever, polymyalgia rheumatica
   Reactive arthritis
   Inflammatory soft-tissue diseases of other origins
   Arthritic symptoms in the case of degenerative joint diseases (arthroses)
   Traumatic arthritides
   Collagenoses of any origin, e.g., systemic lupus erythematodes, sclerodermia, polymyositis, dermatomyositis, Sjögren's syndrome, Still's syndrome, Felty's syndrome
(iii) Allergies that are accompanied by inflammatory and/or proliferative processes:
   All forms of allergic reactions, e.g., Quincke's edema, hay fever, insect bites, allergic reactions to pharmaceutical agents, blood derivatives, contrast media, etc., anaphylactic shock, urticaria, contact dermatitis
(iv) Vascular inflammations (vasculitides)
   Panarteritis nodosa, temporal arteritis, erythema nodosum
(v) Dermatological diseases that are accompanied by inflammatory, allergic and/or proliferative processes:
   Atopic dermatitis (primarily in children)
   Psoriasis
   Pityriasis rubra pilaris
   Erythematous diseases, triggered by different noxae, e.g., radiation, chemicals, burns, etc.
   Bullous dermatoses
   Diseases of the lichenoid group
   Pruritis (e.g., of allergic origin)
   Seborrheal eczema
   Rosacea
   Pemphigus vulgaris
   Erythema exudativum multiforme
   Balanitis
   Vulvitis
   Hair loss such as alopecia areata
   Cutaneous T-cell lymphoma
(vi) Kidney diseases that are accompanied by inflammatory, allergic and/or proliferative processes:
   Nephrotic syndrome
   All nephritides
(vii) Liver diseases that are accompanied by inflammatory, allergic and/or proliferative processes:
   Acute liver cell decomposition
   Acute hepatitis of different origins, e.g., viral, toxic, pharmaceutical agent-induced
   Chronic aggressive hepatitis and/or chronic intermittent hepatitis
(viii) Gastrointestinal diseases that are accompanied by inflammatory, allergic and/or proliferative processes:
   Regional enteritis (Crohn's disease)
   Colitis ulcerosa
   Gastritis
   Reflux esophagitis
   Ulcerative colitis of other origins, e.g., native sprue
(ix) Proctologic diseases that are accompanied by inflammatory, allergic and/or proliferative processes:
   Anal eczema
   Fissures
   Hemorrhoids
   Idiopathic proctitis
(x) Eye diseases that are accompanied by inflammatory, allergic and/or proliferative processes:
   Allergic keratitis, uveitis, iritis
   Conjunctivitis
   Blepharitis
   Optic neuritis
   Chorioiditis
   Sympathetic ophthalmia
(xi) Diseases of the ear-nose-throat area that are accompanied by inflammatory, allergic and/or proliferative processes:
   Allergic rhinitis, hay fever
   Otitis externa, e.g., caused by contact dermatitis, infection, etc.
   Otitis media
(xii) Neurological diseases that are accompanied by inflammatory, allergic and/or proliferative processes:
   Cerebral edema, primarily tumor-induced cerebral edema
   Multiple sclerosis
   Acute encephalomyelitis
   Meningitis
   Various forms of convulsions, e.g., infantile nodding spasms
(xiii) Blood diseases that are accompanied by inflammatory, allergic and/or proliferative processes:
   Acquired hemolytic anemia
   Idiopathic thrombocytopenia
(xiv) Tumor diseases that are accompanied by inflammatory, allergic and/or proliferative processes:
   Acute lymphatic leukemia
   Malignant lymphoma
   Lymphogranulomatoses
   Lymphosarcoma
   Extensive metastases, mainly in breast, bronchial and prostate cancers
(xv) Endocrine diseases that are accompanied by inflammatory, allergic and/or proliferative processes:
   Endocrine orbitopathy
   Thyreotoxic crisis
   De Quervain's thyroiditis
   Hashimoto's thyroiditis
   Basedow's disease
(xvi) Organ and tissue transplants, graft-versus-host disease
(xvii) Severe shock conditions, e.g., anaphylactic shock, systemic inflammatory response syndrome (SIRS)
(xviii) Vomiting that is accompanied by inflammatory, allergic and/or proliferative processes:
   e.g., in combination with a 5-HT3 antagonist in cytostatic-agent-induced vomiting
(xix) Pain of inflammatory origin, e.g., lumbago
(xx) Substitution therapy in:
   Innate primary suprarenal insufficiency, e.g., congenital adrenogenital syndrome
   Acquired primary suprarenal insufficiency, e.g., Addison's disease, autoimmune adrenalitis, meta-infective tumors, metastases, etc.
   Innate secondary suprarenal insufficiency, e.g., congenital hypopituitarism
   Acquired secondary suprarenal insufficiency, e.g., meta-infective tumors, etc.

Pharmaceutical agents that contain stereoisomers of general formula I show special effectiveness in the case of the following diseases:
1. Lung diseases
2. Rheumatic diseases/autoimmune diseases
3. Dermatological diseases
4. Degenerative joint diseases
5. Vascular inflammations
6. Graft-versus-host disease
7. Severe shock conditions
8. Vomiting that is accompanied by inflammatory, allergic and/or proliferative processes
9. Inflammation-induced pain.

Moreover, the compounds of general formula (I) according to the invention can be used for treatment and prophylaxis of additional pathologic conditions that are not mentioned above, for which synthetic glucocorticoids are now used (see in this respect Hatz, H. J., Glucocorticoide: Immunologische Grundlagen, Pharmakologie und Therapierichtlinien, Wissenschaftliche Verlagsgesellschaft mbH, Stuttgart, 1998).

All previously mentioned indications are described in detail in Hatz, H. J., Glucocorticoide: Immunologische Grundlagen, Pharmakologie und Therapierichtlinien, Wissenschaftliche Verlagsgesellschaft mbH, Stuttgart, 1998.

For the therapeutic actions in the above-mentioned pathologic conditions, the suitable dose varies and depends on, for example, the active strength of the compound of general formula (I), the patient (e.g., size, weight, sex, etc.), the type of administration, and the type and severity of the conditions that are to be treated, as well as the use as a prophylactic agent or therapeutic agent.

The invention relates to the use of the claimed compounds for the production of a pharmaceutical composition.

In addition, the invention provides:
(i) The use of one of the compounds of general formula (I) according to the invention or mixture thereof for the production of a pharmaceutical composition for treating or preventing inflammatory processes, and in particular for treating a DISEASE (as defined above);
(ii) A process for treating or preventing inflammatory processes, in particular for treating a DISEASE (as defined above), said process comprises an administration of a pharmaceutically effective amount of a compound of general formula (I), whereby this amount reduces or suppresses the disease or the symptoms, and whereby the compound is given to a patient, preferably a mammal, preferably a human, who requires such a treatment;
(iii) A pharmaceutical composition anti-inflammatory action, in particular for treating a DISEASE (as defined above), whereby the composition comprises one of the compounds according to the invention or mixture thereof and optionally at least one pharmaceutical adjuvant and/or vehicle.

In general, satisfactory results can be expected in animals when the daily doses comprise a range of 1 μg to 100,000 μg of the compound according to the invention per kg of body weight. In the case of larger mammals, such as, for example, the human, a recommended daily dose lies in the range of 1 μg to 100,000 μg per kg of body weight. Preferred is a dose of 10 to 30,000 μg per kg of body weight, and more preferred is a dose of 10 to 10,000 μg per kg of body weight. For example, this dose is suitably administered several times daily. For treating acute shock (e.g., anaphylactic shock), individual doses can be given that are significantly above the above-mentioned doses.

The formulation of the pharmaceutical preparations based on the new compounds is carried out in a way that is known in the art by the active ingredient being processed with the vehicles, fillers, substances that influence decomposition, binding agents, moisturizers, lubricants, absorbents, diluents, flavoring correctives, coloring agents, etc., that are commonly used in galenicals and converted into the desired form of administration. In this case, reference is made to Remington's Pharmaceutical Science, 15^{th} Edition, Mack Publishing Company, East Pennsylvania (1980).

For oral administration, especially tablets, coated tablets, capsules, pills, powders, granulates, lozenges, suspensions, emulsions or solutions are suitable.

For parenteral administration, injection and infusion preparations are possible.

For intra-articular injection, correspondingly prepared crystal suspensions can be used.

For intramuscular injection, aqueous and oily injection solutions or suspensions and corresponding depot preparations can be used.

For rectal administration, the new compounds can be used in the form of suppositories, capsules, solutions (e.g., in the form of enemas) and ointments both for systemic and for local treatment.

For pulmonary administration of the new compounds, the latter can be used in the form of aerosols and inhalants.

For local application to eyes, outer ear channels, middle ears, nasal cavities, and paranasal sinuses, the new compounds can be used as drops, ointments, tinctures and gels in corresponding pharmaceutical preparations.

For topical application, formulations in gels, ointments, fatty ointments, creams, pastes, powders, suspensions, emulsions and solutions are possible. The dosage of the compounds of general formula (I) should be 0.01%/-20% in these preparations to achieve a sufficient pharmacological action.

The invention also comprises the compounds of general formula (I) according to the invention as therapeutic active ingredients. In addition, the compounds of general formula (I) according to the invention are part of the invention as therapeutic active ingredients together with one or more pharmaceutically compatible and acceptable adjuvants and/or vehicles.

The compounds of general formula (I) according to the invention can optionally also be formulated and/or administered in combination with other active ingredients.

The invention therefore also relates to combination therapies or combined compositions, in which a compound of general formula (I) or a pharmaceutically acceptable salt thereof, or a pharmaceutical composition that contains a compound of general formula (I) or a pharmaceutically acceptable salt thereof, is administered either simultaneously (optionally in the same composition) or in succession together with one or more pharmaceutical agents for treating one of the above-mentioned pathologic conditions. For example, for treatment of rheumatoid arthritis, osteoartritis, COPD (chronic obstructive lung disease), asthma or allergic rhinitis, a compound of general formula (I) of this invention can be combined with one or more pharmaceutical agents for treating such a condition. When such a combination is administered by inhalation, the pharmaceutical agent that is to be combined can be selected from the following list:
A PDE4 inhibitor including an inhibitor of the PDE4D isoform,
A selective β.sub2.adrenoceptor agonist, such as, for example, metaproterenol, isoproterenol, isoprenaline, albuterol, salbutamol, formoterol, salmeterol, terbutaline, orcipresnaline, bitolterol mesylate, pirbuterol or indacaterol;
A muscarine receptor antagonist (for example, an M1, M2 or M3 antagonist, such as, for example, a more selective M3 antagonist), such as, for example, ipratropium bromide, tiotropium bromide, oxitropium bromide, pirenzepine or telenzepine;
A modulator of the chemokine receptor function (such as, for example, a CCR1 receptor antagonist); or
An inhibitor of the p38 kinase function.

For another subject of this invention, such a combination with a compound of general formula (I) or a pharmaceutically acceptable salt thereof is used for treatment of COPD, asthma or allergic rhinitis and can be administered by inhalation or orally in combination with xanthine (such as, for example, aminophylline or thyeophylline), which also can be administered by inhalation or orally.

### EXAMPLES

### Synthesis 1: Production of Tetralone Derivatives of General Formula (II)

### A. 6-Chloro-2-hydroxy-5-methoxy-4,4-dimethyl-2-trifluoromethyl-3,4-dihydro-2H-naphthalen-1-one

3 g of 4-(3-chloro-2-methoxyphenyl)-2-hydroxy-4-methyl-2-trifluoromethyl-pentanal, dissolved in dichloromethane (DCM), is mixed with 30 ml of trifluoroacetic acid and stirred for 2 days at room temperature. The reaction is worked up in aqueous form, and the crude product is chromatographed on silica gel. The desired isomeric diols are obtained in a 60% total yield.

1 g of diol (3.1 mmol) in DCM is mixed at 0° C. with 31 mmol of SO₃-pyridine complex, 52 mmol of triethylamine, and 25 mmol of DMSO are stirred for 3 days at room temperature. Working-up with DCM/NH₄Cl solution and chromatography on silica gel yields 600 mg of the desired tetralone. ¹H-NMR (300 MHz, CDCl₃): 1.48 (s, 3H), 1.66 (s, 3H), 2.16 (dd, 1H), 2.47 (dd, 1H), 3.94 (s, 3H), 7.40 (d, 1H), 7.19 (d, 1H).

### B. 2-Hydroxy-7-methoxy-4,4-dimethyl-2-trifluoromethyl-3,4-dihydro-2H-naphthalen-1-one

Analogously to the synthesis that is described under A, the title compound is produced starting from 2-hydroxy-4-(4-methoxyphenyl)-4-methyl-2-trifluoromethyl-pentanal. ¹H-NMR (300 MHz, CDCl₃): 1.29 (s, 3H), 1.50 (s, 3H), 2.19 (dd, 1H), 2.40 (dd, 1H), 3.84 (s, 3H), 4.05 (s, 1H), 7.19 (dd, 1H), 7.32 (m, 2H).

### Example 1

### Reaction of Compounds of General Formula (ID with Nucleophilic Reagents of General Formula R³—X-M with the Formation of Compounds of General Formula (I)

### 1-Benzyl-6-chloro-5-methoxy-4,4-dimethyl-2-trifluoromethyl-1,2,3,4-tetrahydronaphthalene-1,2-diol

0.31 mmol of 6-chloro-2-hydroxy-5-methoxy-4,4-dimethyl-2-trifluoromethyl-3,4-dihydro-2H-naphthalen-1-one was mixed at −75° C. in THF with 1.2 mmol of benzylmagnesium bromide (1 M in diethyl ether). The reaction mixture is brought to room temperature overnight; the reaction is completed by adding NH₄Cl solution, extracted with dichloromethane, washed with NaCl solution, and the crude product is chromatographed on silica gel. 30% of the title compound is obtained.

1H-NMR (300 MHz, CDCl₃): δ/ppm=1.51 (s, 6H), 2.82 (d, 1H), 3.41 (d, 1H), 3.93 (s, 3H), 6.34 (d, 1H), 7.02 (d, 1H), 7.10-7.45 (m, 7H).

### Example 2

### Reaction of Compounds of General Formula (II) with Nucleophilic Reagents of General Formula Reactive Group)-X-M to Form an Intermediate Product

### A. 6—Chloro-5-methoxy-2,4,4-trimethyl-1-vinyl-1,2,3,4-tetrahydro-naphthalene-1,2-diol

0.31 mmol of 6-chloro-2-hydroxy-5-methoxy-4,4-dimethyl-2-trifluoromethyl-3,4-dihydro-2H-naphthalen-1-one, dissolved in THF, is mixed at −75° C. with 0.93 mmol of vinylmagnesium bromide (1 M solution in THF), and it is thawed overnight. The reaction is completed by adding NH₄Cl solution, extracted with ethyl acetate, washed with NaCl solution, and the crude product is chromatographed on silica gel. 90% of the title compound is obtained.

¹H-NMR (300 MHz, CDCl₃): δ/ppm=1.51 (s, 3H), 1.58 (s, 3H), 2.00 (d, 1H), 2.12 (s, 1H), 2.36 (s, 1H), 2.47 (d, 1H), 3.94 (s, 3H), 5.32 (dd, 1H), 5.47 (dd, 1H), 6.30 (dd, 1H), 7.19 (d, 1H), 7.28 (d, 1H).

### B. 1-Allyl-6-chloro-5-methoxy-4,4-dimethyl-2-trifluoromethyl-1,2,3,4-tetrahydronaphthalene-1,2-diol

0.26 mmol of 6-chloro-2-hydroxy-5-methoxy-4,4-dimethyl-2-trifluoromethyl-3,4-dihydro-2H-naphthalen-1-one, dissolved in THF, is mixed at −75° C. with 1.25 mmol of allylylmagnesium bromide (1 M solution in THF), and it is thawed overnight. The reaction is completed by adding NH₄Cl solution, extracted with ethyl acetate, washed with NaCl solution, and the crude product is chromatographed on silica gel. The two diastereomers that are formed are also separated in this case. The desired allyl compound is obtained with a 50% total yield.

¹H-NMR (300 MHz, CDCl₃): Diastereomer A: δ/ppm=1.49 (s, 3H), 1.60 (s, 3H), 2.01 (d, 1H), 2.19 (d, 1H), 2.60 (s, 1H), 2.70 (m, 2H), 3.36 (s, 1H), 3.94 (s, 3H), 5.04 (m, 2H), 5.45 (m, 1H), 7.29 (m, 2H); Diastereomer B: δ/ppm=1.51 (s, 3H), 1.53 (s, 3H), 2.15 (dd, 1H), 2.28 (s, 1H), 2.35 (dd, 1H), 2.54 (s, 1H), 2.88 (m, 2H), 2.94 (s, 1H), 3.91 (s, 3H), 5.21 (m, 2H), 5.59 (m, 1H), 7.07 (d, 1H), 7.25 (d, 1H).

C. Analogously to Example 2B, 1-allyl-7-methoxy-4,4-dimethyl-2-trifluoromethyl-1,2,3,4-tetrahydronaphthalene-1,2-diol is produced as a diastereomer mixture.

Main Isomer: ¹H-NMR (300 MHz, CDCl₃): δ/ppm=1.38 (s, 3H), 1.39 (s, 3H), 2.11 (m, 1H), 2.30 (s, 1H), 2.40 (m, 1H), 2.49 (s, 2H), 2.90 (dd, 1H), 2.98 (s, 1H), 3.81 (s, 3H), 5.21 (m, 2H), 5.69 (m, 1H), 6.83 (d, 1H), 7.21 (d, 1H).

### D. 1-Ethynyl-7-methoxy-4,4-dimethyl-2-trifluoromethyl-1,2,3,4-tetrahydronaphthalene-1.2-diol

0.35 mmol of 2-hydroxy-7-methoxy-4,4-dimethyl-2-trifluoromethyl-3,4-dihydro-2H-naphthalen-1one is introduced into THF at −75° C. and mixed with 1.03 mmol of trimethylsilyl-alkinyl-lithium. The reaction mixture is stirred for 30 minutes, thawed, and the reaction is completed by adding phosphate buffer solution (pH 7). The mixture is extracted with ethyl acetate, washed with NaCl solution, dried with sodium sulfate, and the crude product is chromatographed on silica gel: 100 mg of the TMS-acetylene compound.

¹H-NMR (300 MHz, CDCl₃): δ/ppm=0.22 (s, 9H), 1.34 (s, 3H), 1.44 (s, 3H), 2.08 (d, 1H), 2.32 (d, 1H), 3.81 (s, 3H), 5.93 (dd, 1H), 7.30 (d, 1H), 7.43 (d, 1H); MS (ESI): 369 (M−H₂O+1).

After acidic working-up, reaction with tetrabutylammonium fluoride in THF yields 1-ethynyl-7-methoxy-4,4-dimethyl-2-trifluoromethyl-1,2,3,4-tetrahydronaphthalene-1,2-diol.

MS (CI/NH₃): 314, 332 (M, M+18).

### Example 3

### Modification of the Reactive Group After Introduction into the Compound of General Formula (In to Obtain an Additional Intermediate Product

### 6-Chloro-5-methoxy-2,4,4-trimethyl-1,2,3,4-tetrahydro-naphthalene-1,2-diol-1-carbaldehyde

0.25 mmol of the vinyl compound of Example 2A is ozonized into DCM for 5-10 minutes at −75° C. 2.75 mmol of dimethyl sulfide is added, it is stirred for 2 more hours, and the mixture is allowed to come to room temperature in this case. It is evaporated to the dry state and chromatographed on silica gel. The corresponding aldehyde is obtained in a 45% yield.

¹H-NMR (300 MHz, CDCl₃): δ/ppm=1.52 (s, 3H), 1.67 (s, 3H), 1.96 (d, 1H), 2.48 (s, 1H), 2.52 (d, 1H), 3.96 (s, 3H), 4.37 (s, 1H), 6.55 (d, 1H), 7.27 (d, 1H), 9.88 (s, 1H).

### Example 4

### Production of Compounds of General Formula (I)

### 6-Chloro-5-methoxy-2,4,4-trimethyl-1-[(2-methyl-quinolin-5-ylamino)-methyl]-1,2,3,4-tetrahydro-naphthalene-1,2-diol

A. 0.12 mmol of the aldehyde of Example 3 is stirred in toluene with 0.16 mmol of 2-methyl-5-aminoquinoline and 0.3 mmol of titanium tetraethylate for 5 hours at 120° C. After cooling, it was quenched with NaHCO₃ solution, suctioned off on diatomaceous earth, dried, concentrated by evaporation and chromatographed. The corresponding imine is obtained in an 80% yield.

¹H-NMR (300 MHz, CDCl₃): δ/ppm=1.60 (s, 3H), 1.19 (s, 3H), 2.02 (d, 1H), 2.69 (d, 1H), 2.80 (s, 3H), 3.99 (s, 3H), 5.50 (s, 1H), 6.89 (d, 1H), 7.08 (d, 1H), 7.30(d, 1H), 7.40 (d, 1H), 7.68 (dd, 1H), 8.02 (d, 1H), 8.41 (s, 1H), 8.51 (d, 1H).

30 mg of the imine in methanol is mixed with some acetic acid and hydrogenated under hydrogen atmosphere with palladium on activated carbon as a catalyst for 2 hours. It is suctioned off, concentrated by evaporation and recrystallized from ether/hexane. 7 mg of the title compound is obtained.

B. Analogously to the reaction that is described under A, the following imino compound is obtained:

7-Methoxy-4,4-dimethyl-1-[2-methyl-quinolin-5-ylimino]-methyl}-2-trifluoromethyl-1,2,3,4-tetrahydronaphthalene-1,2-diol.

¹H-NMR (300 MHz, CDCl₃): δ/ppm=1.46 (s, 3H), 1.55 (s, 3H), 2.08 (d, 1H), 2.65 (d, 1H), 2.73 (s, 1H), 2.79 (s, 3H), 3.72 (s, 3H), 5.43 (s, 1H), 6.70 (d, 1H), 6.95 (dd, 1H), 7.08 (dd, 1H), 7.36 (d, 1H), 7.42 (d, 1H), 7.65 (t, 1H), 7.94 (d, 1H), 8.45 (m, 2H); (MS (ESI): 461 (M+1)).

This imino compound is reduced to the following compound of general formula (I) analogously to the reaction that is described under A:

7-Methoxy-4,4-dimethyl-1-[(2-methyl-quinolin-5-ylamino)methyl]-2-trifluoromethyl-1,2,3,4-tetrahydronaphthalene-1,2-diol.

### Example 5

### Modification of the R^{3a} Group in Compounds of General Formula (I)

### A. 1-Benzyl-6-chloro-5-methoxy-4,4-dimethyl-2-trifluoromethyl-1,2,3 4-tetrahydro-naphthalen-2-ol

The title compound is obtained by benzylic deoxygenation of the compound of Example 1 (e.g., by reaction with triethylsilane/boron trifluoride-etherate according to Chem. Eur. J. 2001, 7, 993-1005).

B. The following compound is obtained analogously from the amino compound of Example 4B:

7-Methoxy-4,4-dimethyl-1-[(2-methyl-quinolin-5-ylamino)-methyl]-2-trifluoromethyl-1,2,3,4-tetrahydronaphthalen-2-ol.

By means of the process described above by way of example, the following compounds can be produced analogously, whereby corresponding starting compounds (amines and aldehydes used) are described in WO 2005/003098 and WO 2005/034939 and subsequent reactions (ether cleavage and synthesis of indoles or azaindoles from corresponding alkines) are described in WO 2003/082280 and WO 2003/082827:
6-Chloro-5-methoxy-4,4-dimethyl-1-{[(2-methylquinolin-5-yl)amino]methyl}-2-(trifluoromethyl)-1,2,3,4-tetrahydronaphthalene-1,2-diol
7-Methoxy-4,4-dimethyl-1-{[(2-methylquinolin-5-yl)amino]methyl}-2-(trifluoromethyl)-1,2,3,4-tetrahydronaphthalene-1,2-diol
1-Benzyl-6-chloro-5-methoxy-4,4-dimethyl-2-(trifluoromethyl)-1,2,3,4-tetrahydronaphthalene-1,2-diol
6-Chloro-5-methoxy-4,4-dimethyl-1-{[(2-methylquinazolin-5-yl)amino]methyl}-2-(trifluoromethyl)-1,2,3,4-tetrahydronaphthalene-1,2-diol
6-Chloro-1-{[(7-fluoro-2-methylquinazolin-5-yl)amino]methyl}-5-methoxy-4,4-dimethyl-2-(trifluoromethyl)-1,2,3,4-tetrahydronaphthalene-1,2-diol
6-Chloro-1-{[(8-fluoro-2-methylquinazolin-5-yl)amino]methyl}-5-methoxy-4,4-dimethyl-2-(trifluoromethyl)-1,2,3,4-tetrahydronaphthalene-1,2-diol
6-Chloro-1-{[(7,8-difluoro-2-methylquinazolin-5-yl)amino]methyl}-5-methoxy-4,4-dimethyl-2-(trifluoromethyl)-1,2,3,4-tetrahydronaphthalene-1,2-diol
5-{[(6-Chloro-1,2-dihydroxy-5-methoxy-4,4-dimethyl-2-trifluoromethyl-1,2,3,4-tetrahydronaphthalen-1-yl)methyl]amino}quinolin-2(1H)-one
5-{[(6-Chloro-1,2-dihydroxy-5-methoxy-4,4-dimethyl-2-trifluoromethyl-1,2,3,4tetrahydronaphthalen-1-yl)methyl]amino}-2-methylphthalazin-1-one
6-Chloro-4,4-dimethyl-1-{[(2-methylquinolin-5-yl)amino]methyl)}-2-(trifluoromethyl)-1,2,3,4-tetrahydronaphthalene-1,2,5-triol
6-Chloro-4,4-dimethyl-1-{[(2-methylquinazolin-5-yl)amino]methyl}-2-(trifluoromethyl)-1,2,3,4-tetrahydronaphthalene-1,2,5-triol
6-Chloro-1-{[(7-fluoro-2-methylquinazolin-5-yl)amino]methyl}-4,4-dimethyl-2-(trifluoromethyl)-1,2,3,4-tetrahydronaphthalene-1,2,5-triol
6-Chloro-1-{[(8-fluoro-2-methylquinazolin-5-yl)amino]methyl}-4,4-dimethyl-2-(trifluoromethyl)-1,2,3,4-tetrahydronaphthalene-1,2,5-triol
6-Chloro-1-{[(7,8-difluoro-2-methylquinazolin-5-yl)amino]methyl}-4,4-dimethyl-2-(trifluoromethyl)-1,2,3,4-tetrahydronaphthalene-1,2,5-triol
5-{[(6-Chloro-1,2,5-trihydroxy-4,4-dimethyl-2-trifluoromethyl-1,2,3,4-tetrahydronaphthalen-1-yl)methyl]amino}quinolin-2(1H)-one
5-{[(6-Chloro-1,2,5-trihydroxy-4,4-dimethyl-2-trifluoromethyl-1,2,3,4-tetrahydronaphthalen-1-yl)methyl]amino}-8-fluoroquinolin-2(1H)-one
5-{[(6-Chloro-1,2,5-trihydroxy-4,4-dimethyl-2-trifluoromethyl-1,2,3,4-tetrahydronaphthalen-1-yl)methyl]amino}isoquinolin-1(2H)-one
5-{[(6-Chloro-1,2,5-trihydroxy-4,4-dimethyl-2-trifluoromethyl-1,2,3,4-tetrahydronaphthalen-1-yl)methyl]amino}-2-methylphthalazin-1-one
6-Chloro-4,4-dimethyl-1-{[(2-methylquinolin-5-yl)amino]methyl}-2-trifluoromethyl-1,2,3,4-tetrahydronaphthalene-2,5-diol
7-Methoxy-4,4-dimethyl-1-{[(2-methylquinolin-5-yl)amino]methyl}-2-(trifluoromethyl)-1,2,3,4-tetrahydronaphthalen-2-ol
6-Chloro-4,4-dimethyl-1-{[(2-methylquinazolin-5-yl)amino]methyl}-2-(trifluoromethyl)-1,2,3,4-tetrahydronaphthalene-2,5-diol
6-Chloro-1-{[(7-fluoro-2-methylquinazolin-5-yl)amino]methyl}-4,4-dimethyl-2-(trifluoromethyl)-1,2,3,4-tetrahydronaphthalene-2,5-diol
6-Chloro-1-{[(8-fluoro-2-methylquinazolin-5-yl)amino]methyl}-4,4-dimethyl-2-(trifluoromethyl)-1,2,3,4-tetrahydronaphthalene-2,5-diol
6-Chloro-1-{[(7,8-difluoro-2-methylquinazolin-5-yl)amino]methyl)}-4,4-dimethyl-2-(trifluoromethyl)-1,2,3,4-tetrahydronaphthalene-2,5-diol
5-{[(6-Chloro-2,5-dihydroxy-4,4-dimethyl-2-trifluoromethyl-1,2,3,4-tetrahydronaphthalen-1-yl)methyl]amino}quinolin-2(1H)-one
5-{[(6-Chloro-2,5-dihydroxy-4,4-dimethyl-2-trifluoromethyl-1,2,3,4-tetrahydronaphthalen-1-yl)methyl]amino}-8-fluoroquinolin-2(1H)-one
5-{[(6-Chloro-2,5-dihydroxy-4,4-dimethyl-2-trifluoromethyl-1,2,3,4-tetrahydronaphthalen-1-yl)methyl]amino}isoquinolin-1(2B)-one
5-{[(6-Chloro-2,5-dihydroxy-4,4-dimethyl-2-trifluoromethyl-1,2,3,4-tetrahydronaphthalen-1-yl)methyl]amino-}2-methylphthalazin-1-one
6-Fluoro-5-methoxy-4-ethyl-1-{[(2-methylquinolin-5-yl)amino]methyl}-2-(trifluoromethyl)-1,2,3,4-tetrahydronaphthalene-1,2-diol
6-Fluoro-1-{[(8-fluoro-2-methylquinazolin-5-yl)amino]methyl}-5-methoxy-4-ethyl-2-(trifluoromethyl-1,2,3,4-tetrahydronaphthalene-1,2-diol
5-{[(6-Fluoro-1,2-dihydroxy-4-ethyl-5-methoxy-2-trifluoromethyl-1,2,3,4-tetrahydronaphthalen-1-yl)methyl]amino}quinolin-2-(1H)-one
6-Fluoro-4-ethyl-1-{[(indazol-4-yl)amino]methyl}-5-methoxy-2-(trifluoromethyl)-1,2,3,4-tetrahydronaphthalene-1,2-diol
5-{[(6-Fluoro-1,2-dihydroxy-4-ethyl-5-methoxy-2-[trifluoromethyl]-1,2,3,4-tetrahydronaphthalen-1-yl)methyl]amino}-2-methylphthalazin-1-one
6-Fluoro-4-ethyl-1-{[(2-methylquinolin-5-yl)amino]methyl}-2-(trifluoromethyl)-1,2,3,4-tetrahydronaphthalene-2,5-diol
6-Fluoro-1-{[(8-fluoro-2-methylquinazolin-5-yl)amino]methyl}-4-ethyl-2-(trifluoromethyl)-1,2,3,4-tetrahydronaphthalene-2,5-diol
5-{[(6-Fluoro-2,5-dihydroxy-4-ethyl-2-[trifluoromethyl]-1,2,3,4-tetrahydronaphthalen-1-yl)methyl]amino}quinolin-2(1H)-one
6-Fluoro-4-ethyl-1-{[(indazol-4-yl)amino]methyl}-2-(trifluoromethyl)-1,2,3,4-tetrahydronaphthalene-2,5-diol
5-{[(6-Fluoro-2,5-dihydroxy-4-ethyl-2-[trifluoromethyl]-1,2,3,4-tetrahydronaphthalen-1-yl)methyl]amino}-2-methylphthalazin-1-one
6-Fluoro-5-methoxy-4-ethyl-1-{[(2-methylquinolin-5-yl)amino]methyl}-2-trifluoromethyl-1,2,3,4-tetrahydronaphthalene-1,2-diol
6-Fluoro-1-{[(8-fluoro-2-methylquinazolin-5-yl)amino]methyl}-5-methoxy-4-ethyl-2-trifluoromethyl-1,2,3,4-tetrahydronaphthalene-1,2-diol
5-{[(6-Fluoro-1,2-dihydroxy-4-ethyl-5-methoxy-2-trifluoromethyl-1,2,3,4-tetrahydronaphthalen-1-yl)methyl]amino}quinolin-2(1H)-one
6-Fluoro-4-ethyl-1-{[(indazol-4-yl)amino]methyl}-5-methoxy-2-(trifluoromethyl)-1,2,3,4-tetrahydronaphthalene-1,2-diol
5-{[(6-Fluoro-1,2-dihydroxy-4-ethyl-5-methoxy-2-[trifluoromethyl]-1,2,3,4-tetrahydronaphthalen-1-yl)methyl]amino}-2-methylphthalazin-1-one
6-Fluoro-4-ethyl-1-{[(2-methylquinolin-5-yl)amino]methyl}-2-(trifluoromethyl)-1,2,3,4-tetrahydronaphthalene-2,5-diol
6-Fluoro-1-{[(8-fluoro-2-methylquinazolin-5-yl)amino]methyl}-4-ethyl-2-trifluoromethyl-1,2,3,4-tetrahydronaphthalene-2,5-diol
5-{[(6-Fluoro-2,5-dihydroxy-4-ethyl-2-[trifluoromethyl]-1,2,3,4-tetrahydronaphthalen-1-yl)methyl]amino}chromen-2-one
6-Fluoro-4-ethyl-1-{[(indazol-4-yl)amino]methyl}-2-trifluoromethyl-1,2,3,4-tetrahydronaphthalene-2,5-diol
5-{[(6-Fluoro-2,5-dihydroxy-4-ethyl-2-[trifluoromethyl]-1,2,3,4-tetrahydronaphthalen-1-yl)methyl]amino}-2-methylphthalazin-1-one
6-Chloro-1-(1H-indol-2-yl)-5-methoxy-4,4-dimethyl-2-(trifluoromethyl)-1,2,3,4-tetrahydronaphthalene-1,2-diol
6-Chloro-1-(1H-indol-2-yl)-4,4-dimethyl-2-(trifluoromethyl-1,2,3,4-tetrahydronaphthalene-2,5-diol
6-Fluoro-1-(1H-indol-2-yl)-5-methoxy-4,4-dimethyl-2-(trifluoromethyl)-1,2,3,4-tetrahydronaphthalene-1,2-diol
6-Fluoro-1-(1H-indol-2-yl)-4,4-dimethyl-2-(trifluoromethyl)-1,2,3,4-tetrahydronaphthalene-2,5-diol
6-Chloro-5-methoxy-4,4-dimethyl-1-(1H-pyrrolo [3,2-c]pyridin-2-yl)-2-(trifluoromethyl)-1,2,3,4-tetrahydronaphthalene-1,2-diol
6-Chloro-4,4-dimethyl-1-(1H-pyrrolo[3,2-c]pyridin-2-yl)-2-(trifluoromethyl)-1,2,3,4-tetrahydronaphthalene-2,5-diol
6-Fluoro-5-methoxy-4,4-dimethyl-1-(1H-pyrrolo[3,2-c]pyridin-2-yl)-2-(trifluoromethyl)-1,2,3,4-tetrahydronaphthalene-1,2-diol
6-Fluoro-4,4-dimethyl-1-(1H-pyrrolo[3,2-c]pyridin-2-yl)-2-(trifluoromethyl)-1,2,3,4-tetrahydronaphthalene-2,5-diol
5-{[(6,7-Dihydroxy-9,9-dimethyl-7-trifluoromethyl-6,7,8,9-tetrahydronaphtho[1,2-d][1,3]dioxol-6-yl)methyl]amino}quinolin-2(1H)-one
6-{[(Indazol-4-yl)amino]methyl}-9,9-dimethyl-7-trifluoromethyl-6,7,8,9-tetrahydronaphtho[1,2-d][1,3]dioxole-6,7-diol
5-{[(6,7-Dihydroxy-9,9-dimethyl-7-trifluoromethyl-6,7,8,9-tetrahydronaphtho[1,2-d][1,3]dioxol-6-yl)methyl]amino}-2-methylphthalazin-1-one
6-{[(2-Methylquinolin-5-yl)amino]methyl}-9,9-dimethyl-7-(trifluoromethyl)-6,7,8,9-tetrahydronaphtho[1,2-d][1,3]dioxole-6,7-diol
6-{[(8-Fluoro-2-methylquinazolin-5-yl)amino]methyl}-9,9-dimethyl-7-(trifluoromethyl)-6,7,8,9-tetrahydronaphtho[1,2-d][1,3]dioxole-6,7-diol
5-{[(7-Hydroxy-9,9-dimethyl-7-trifluoromethyl-6,7,8,9-tetrahydronaphtho[1,2-d][1,3]dioxol-6-yl)methyl]amino}quinolin-2[1H]-one
6-{[(Indazol-4-yl)amino]methyl}-9,9-dimethyl-7-trifluoromethyl-6,7,8,9-tetrahydronaphtho[1,2-d][1,3]dioxol-7-ol
5-{[(7-Hydroxy-9,9-dimethyl-7-trifluoromethyl-6,7,8,9-tetrahydronaphtho[1,2-d][1,3]dioxol-6-yl)methyl]amino}-2-methylphthalazin-1-one
6-{[(2-Methylquinolin-5-yl)amino]methyl}-9,9-dimethyl-7-trifluoromethyl-6,7,8,9-tetrahydronaphtho[1,2-d][1,3]dioxol-7-ol
6-{[(8-Fluoro-2-methylquinazolin-5-yl)amino]methyl}-9,9-dimethyl-7-(trifluoromethyl)-6,7,8,9-tetrahydronaphtho[1,2-d][1,3]dioxol-7-ol
9,9-Dimethyl-1-(1H-pyrrolo[2,3-c]pyridin-2-yl)-7-trifluoromethyl-6,7,8,9-tetrahydronaphtho[1,2-d][1,3]dioxole-6,7-diol
9,9-Dimethyl-1-(1H-pyrrolo[2,3-c]pyridin-2-yl)-7-trifluoromethyl-6,7,8,9-tetrahydronaphtho[1,2-d][1,3]dioxol-7-ol
8-Fluoro-5-{[(6,7-dihydroxy-9,9-dimethyl-7-trifluoromethyl-2,3,6,7,8,9-hexahydronaphtho[1,2-b]furan-6-yl)methyl]amino}quinolin-2(1H)-one
4-{[(6,7-Dihydroxy-9,9-dimethyl-7-trifluoromethyl-2,3,6,7,8,9-hexahydronaphtho[1,2-d]furan-6-yl)methyl]amino}-1,3-dihydroindol-2-one
5-{[(6,7-Dihydroxy-9,9-dimethyl-7-trifluoromethyl-2,3,6,7,8,9-hexahydronaphtho[1,2-d]furan-6-yl)methyl]amino}-2-methylphthalazin-1-one
6-{[(2-Methylquinolin-5-yl)amino]methyl}-9,9-dimethyl-7-(trifluoromethyl)-2,3,6,7,8,9-hexahydronaphtho[1,2-b]furan-6,7-diol
6-{[(7-Fluoro-2-methylquinazolin-5-yl)amino]methyl}-9,9-dimethyl-7-(trifluoromethyl)-2,3,6,7,8,9-hexahydronaphtho[1,2-b]furan-6,7-diol
8-Fluoro-5-{[(7-hydroxy-9,9-dimethyl-7-trifluoromethyl-2,3,6,7,8,9-hexahydronaphtho[1,2-b]furan-6-yl)methyl]amino}quinolin-2(1H)-one
4-{[(7-Hydroxy-9,9-dimethyl-7-trifluoromethyl-2,3,6,7,8,9-hexahydronaphtho[1,2-d]furan-6-yl)methyl]amino}-1,3-dihydroindol-2-one
5-{[(7-Hydroxy-9,9-dimethyl-7-trifluoromethyl-2,3,6,7,8,9-hexahydronaphtho[1,2-b]furan-6-yl)methyl]amino}-2-methylphthalazin-1-one
6-{[(2-Methylquinolin-5-yl)amino]methyl}-9,9-dimethyl-7-(trifluoromethyl)-6,7,8,9-tetrahydronaphtho[1,2-d][1,3]dioxol-7-ol
6-{[(7-Fluoro-2-methylquinazolin-5-yl)amino]methyl}-9,9-dimethyl-7-(trifluoromethyl)-2,3,6,7,8,9-hexahydronaphtho[1,2-b]furan-7-ol
1-(1H-indol-2-yl)-9,9-dimethyl-7-trifluoromethyl-2,3,6,7,8,9-hexahydronaphtho[1,2-b]furan-6,7-diol
9,9-Dimethyl-1-(1H-indol-2-yl)-7-trifluoromethyl)-6,7,8,9-tetrahydronaphtho[1,2-b]furan-7-ol

Without further elaboration, it is believed that one skilled in the art can, using the preceding description, utilize the present invention to its fullest extent. The preceding preferred specific embodiments are, therefore, to be construed as merely illustrative, and not limitative of the remainder of the disclosure in any way whatsoever.

In the foregoing and in the examples, all temperatures are set forth uncorrected in degrees Celsius, and all parts and percentages are by weight, unless otherwise indicated.

The entire disclosures of all applications, patents and publications, cited herein and of corresponding German Application No. 10 2005 017 286.5, filed Apr. 14, 2005 and U.S. Provisional Application Ser. No. 60/671,064, filed Apr. 14, 2005, are incorporated by reference herein.

The preceding examples can be repeated with similar success by substituting the generically or specifically described reactants and/or operating conditions of this invention for those used in the preceding examples.

From the foregoing description, one skilled in the art can easily ascertain the essential characteristics of this invention and, without departing from the spirit and scope thereof, can make various changes and modifications of the invention to adapt it to various usages and conditions.

1. Compounds of general formula (I), 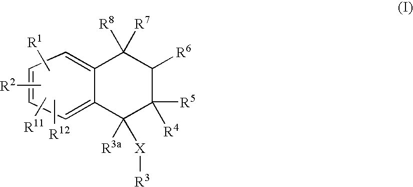
in which
R¹ and R², independently of one another, are a hydrogen atom, a hydroxy group, a halogen atom, an optionally substituted (C₁-C₁₀)-alkyl group, a (C₁-C₁₀)-alkoxy group, a (C₁-C₁₀)-alkylthio group, a (C₁-C₅)-perfluoroalkyl group, a cyano group, a nitro group, or an —NR⁹R^{9a} group,
or R¹ and R² together form a group that is selected from the groups —O—(CH₂)ₙ—O—, —O—(CH₂)ₙ—CH₂—, —O—CH═CH—, —(CH₂)ₙ₊₂—, —NH—(CH₂)ₙ₊₁—, —N(C₁—C₃-alkyl)-(CH₂)ₙ₊₁—, and —NH—N═CH—,
whereby n=1 or 2, and the terminal oxygen atoms and/or carbon atoms and/or nitrogen atoms are linked to directly adjacent ring-carbon atoms,
R¹¹ is a hydrogen atom, a hydroxy group, a halogen atom, a cyano group, an optionally substituted (C₁-C₁₀)-alkyl group, a (C₁-C₁₀)-alkoxy group, a (C₁-C₁₀)-alkylthio group, or a (C₁-C₅)-perfluoroalkyl group,
R¹² is a hydrogen atom, a hydroxy group, a halogen atom, a cyano group, an optionally substituted (C₁-C₁₀)-alkyl group, or a (C₁-C₁₀)-alkoxy group,
R³ is a (C₁-C₁₀)-alkyl group that optionally is substituted by 1 to 3 hydroxy groups, 1 to 3 halogen atoms, and/or 1 to 3 (C₁-C₅)-alkoxy groups, an optionally substituted (C₃-C₇)-cycloalkyl group,
an optionally substituted heterocyclyl group,
an optionally substituted aryl group,
a monocyclic or bicyclic heteroaryl group that optionally is substituted by one or more groups, which are selected, independently of one another, from
(C₁-C₅)-alkyl groups, which themselves optionally can be substituted by 1 to 3 hydroxy groups or 1 to 3 —COOR¹³ groups,
(C₁-C₅)-alkoxy groups,
halogen atoms, hydroxy groups, —NR⁹R^{9a} groups, and
exomethylene groups,
and that contains 1 to 4 nitrogen atoms and/or 1 to 2
oxygen atoms and/or 1 to 2 sulfur atoms and/or 1 to 2 keto groups, whereby this group is linked to the group X via any position, and optionally can be hydrogenated at one or more sites,
R^{3a} means a hydrogen atom, a hydroxy group, an —OR¹⁰ group or an —O(CO)R¹⁰ group,
R⁴ is a hydroxy group, an —OR¹⁰ group or an —O(CO)R¹⁰ group,
R⁵ is a (C₁-C₁₀)-alkyl group, which optionally is partially or completely fluorinated, a (C₃-C₇)cycloalkyl group, a (C₁-C₈)alkyl-(C₃-C₇)cycloalkyl group, a (C₂-C₈)alkenyl-(C₃-C₇)cycloalkyl group, a heterocyclyl group, a (C₁-C₈)alkylheterocyclyl group, a (C₂-C₈)-alkenylheterocyclyl group, an aryl group, a (C₁-C₈)alkylaryl group, a (C₂-C₈)alkenylaryl group, a (C₂-C₈)alkinylaryl-group;
a monocyclic or bicyclic heteroaryl group that optionally is substituted by 1 to 2 keto groups, 1 to 2 (C₁-C₅)-alkyl groups, 1 to 2 (C₁-C₅)-alkoxy groups, 1 to 3 halogen atoms, and/or 1 to 2 exomethylene groups and that contains 1 to 3 nitrogen atoms and/or 1 to 2 oxygen atoms and/or 1 to 2 sulfur atoms;
a (C₁-C₈)alkylheteroaryl group, a (C₂-C₈)alkenylheteroaryl group, or a (C₂-C₈)alkinylheteroaryl group,
whereby this group is linked to the tetrahydronaphthalene system via any position, and optionally can be hydrogenated at one or more sites,
R⁶ is a hydrogen atom, a halogen atom, or an optionally substituted (C₁-C₁₀)-alkyl group,
R⁷ and R⁸, independently of one another, mean a hydrogen atom, a halogen atom, an optionally substituted (C₁-C₁₀)-alkyl group, a cyano group, together a (C₁-C₁₀)-alkylidene group or together with the carbon atom of the tetrahydronaphthalene system an optionally substituted (C₃-C₆)-cycloalkyl ring; or
R⁶ and R⁷ together form an annelated five- to eight-membered, saturated or unsaturated carbocyclic compound or heterocyclic compound, which optionally is substituted by 1 to 2 keto groups, 1 to 2 (C₁-C₅)-alkyl groups, 1 to 2 (C₁-C₅)-alkoxy groups, and/or 1 to 4 halogen atom; or
R¹ and R⁸ together form an annelated five- to eight-membered, saturated or unsaturated carbocyclic compound or heterocyclic compound, which optionally is substituted by 1 to 2 keto groups, 1 to 2 (C₁-C₅)-alkyl groups, 1 to 2 (C₁-C₅)-alkoxy groups, and/or 1 to 4 halogen atoms;
R⁹ and R^{9a}, independently of one another, mean a hydrogen atom, (C₁-C₅)-alkyl or —(CO)—(C₁-C₅)-alkyl,
R₁₀ means a (C₁-C₁₀)-alkyl group or any hydroxy protective group,
R¹³ means a hydrogen atom or a (C₁-C₅)-alkyl group, and
X means a bond or a group —C(═O)—, —C(═S)—, —O—C(═O)—, —O—C(═O)—O—, —O—C(═O)—NH—, —(CH₂)ₚ— (whereby p=1, 2 or 3), a group —(CH₂)ₚ—NH— (whereby p=1, 2 or 3) or a group —NH—, whereby if X contains an NH group, this NH group is connected to substituent R³;
with the condition that if X represents a group —NH—, R^{3a} is not a hydrogen atom; in the form of any stereoisomer or a mixture of stereoisomers, or as a pharmacologically harmless salt or derivative.

2. Compounds according to claim 1, in which
X means a bond or a group —C(═O)—, —C(═S)—, —(CH₂)ₚ— (whereby p=1 or 2), or a group —(CH₂)ₚ—NH— (whereby p=1 or 2).

3. Compounds according to claim 1, whereby
R⁴ is a hydroxy group or a group —OR¹⁰.

4. Compounds according to claim 1, whereby
R⁵ represents a (C₁-C₁₀)-alkyl group, which optionally is partially or completely fluorinated.

5. Compounds according to claim 1, whereby
R⁷ and R⁸ in each case represent a methyl group or form a cyclopropyl group together with the carbon atom of the tetrahydronaphthalene system.

6. Compounds according to claim 1, whereby
R³ means an optionally substituted aryl or heteroaryl group.

7. Compounds according to claim 6, wherein the aryl or heteroaryl group is selected from the group that consists of optionally substituted naphthyl, benzofuranyl, pyrazolo[1,5-a]pyridinyl, phenyl, phthalidyl, isoindolyl, dihydroindolyl, dihydroisoindolyl, dihydroisoquinolinyl, thiophthalidyl, benzoxazinonyl, phthalazinonyl, quinolinyl, isoquinolinyl, chromanyl, isochromanyl, quinolonyl, isoquinolonyl, indazolyl, benzothiazolyl, quinazolinyl, quinoxalinyl, cinnolinyl, phthalazinyl, 1,7- or 1,8-naphthyridinyl, dihydroindolonyl, dihydroisoindolonyl, benzimidazole or indolyl groups.

8. Compounds according to claim 1 for the production of a pharmaceutical agent.

9. Use of a compound according to claim 1 for the production of a pharmaceutical composition for treatment or prevention of inflammatory processes.

10. Process for treatment or prevention of inflammatory processes in a patient, characterized in that a pharmaceutically effective amount of a compound of general formula (I) according to claim 1 is administered to a patient who requires such treatment or prevention.

11. Pharmaceutical preparations that contain at least one compound according to claim 1 as well as one or more pharmaceutically compatible vehicles and/or adjuvants.

12. Process for the production of compounds of general formula (I) according to claim 1, wherein a compound of general formula (II), 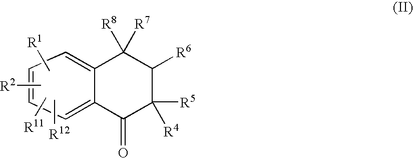
a) is reacted with nucleophilic reagents of general formula R³—X-M, and hydroxy group R^{3a} that is produced is optionally further modified; or
b) is reacted with nucleophilic reagents of general formula (reactive group)-M, and the (reactive group) is converted by further reactions into a group R³—X—, and hydroxy group R^{3a} that is produced is optionally further modified; or
c) is reacted with nucleophilic reagents of general formula R³—NH₂, and hydroxy group R^{3a} that is produced is optionally further modified;
whereby substituents R¹ to R¹² and X have the meanings that are indicated in claim 1, and whereby M is an electrophilic leaving group.

13. Compounds of general formula (II), 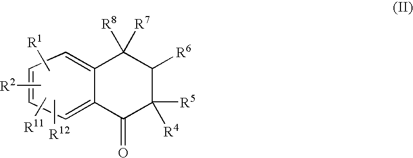
whereby substituents R¹ to R¹² have the meanings that are indicated in claim 1.

14. A method of preparing compounds of general formula (I) according to claim 1 comprising reacting a compound of formula (II).
